(19) United States
(12) Patent Application Publication
(10) Pub. No.: US 2023/0210236 A1
(43) Pub. Date: Jul. 6, 2023

(54) STORAGE DEVICE WITH A MAGNETIC CLOSURE DEVICE

(71) Applicant: Fidlock GmbH, Hannover (DE)

(72) Inventors: Joachim Fiedler, Hannover (DE); Jürgen Spindler, Regensburg (DE); Philip Naftali, Hod Hasharon (IL); Izik Moalem, Hod Hasharon (IL)

(21) Appl. No.: 17/924,843

(22) PCT Filed: May 6, 2021
(86) PCT No.: PCT/EP2021/061939

§ 371 Date: Nov. 11, 2022

(30) Foreign Application Priority Data
May 13, 2020 (DE) 10 2020 206 048.7

(51) Int. Cl. A45C 13/10; A45C 11/22; A45C 13/00; A61L 2/23

(52) U.S. Cl. CPC A45C 13/1069; A45C 11/22; A45C 13/008; A61L 2/23; A61L 2101/12

(57) ABSTRACT

Provided is a storage device including a storage body for holding a liquid, a gas or an item, which has a first wall, a second wall, and a magnetic closure device for closing the storage body, which has a first closure part and a second closure part. The first closure part has a first strip body elongate along a transverse direction and is arranged on a first closing portion of the first wall. The second closure part has a second strip body elongate along a transverse direction and is arranged on a second closing portion of the second wall. The first closure part and the second closure part cooperate in a magnetically attracting manner. At least one of the first wall, the second wall, the first strip body and the second strip body are wholly or partly fabricated from a composition containing an antimicrobially active additive.

### CROSS-REFERENCE TO RELATED APPLICATIONS

This application is the United States national phase of International Application No. PCT/EP2021/061939, filed May 6, 2021, and claims priority to German Patent Application No. 10 2020 206 048.7 filed May 13, 2020, the disclosures of which are hereby incorporated by reference in their entireties.

### BACKGROUND OF THE INVENTION

### Field of the Invention

This disclosure relates to a storage device.

### Description of Related Art

Such a storage device comprises a storage body for holding a liquid, a gas or an item, for example an electronic item such as a mobile phone, a camera of a tablet or laptop computer or the like or also of another item of daily life, for example a purse or a means of payment such as a credit card.

The storage body has a first wall and a second wall, between which the item to be received in the storage device can be stored. The storage device includes a magnetic closure device for closing the storage body, which comprises a first closure part and a second closure part. In one embodiment, the first closure part has a first strip body elongate along a transverse direction and is arranged on a first closing portion of the first wall, and the second closure part has a second strip body elongate along the transverse direction and is arranged on a second closing portion of the second wall. The first closure part and the second closure part cooperate in a magnetically attracting manner in such a way that in a closed position, in which the storage body is closed, the first closing portion and the second closing portion rest against each other.

Such a storage device is known for example from EP 2 571 391 B1.

Such a storage device serves to accommodate items and protect the same, in particular against moisture or dirt or also other external influences. This is to enable items to be carried in difficult environments, such as water sports, but also, for example, at work.

There is a general need to be able to carry and safely handle items in a bag-type storage device.

From US 2015/052617 A1, there is known a security bag for mobile phones, which in addition to electrical shielding can also have antibacterial properties in that the bag is made of a material loaded with silver.

### SUMMARY OF THE INVENTION

It is an object underlying the proposed solution to provide a storage device that enables liquids, gases or items, in particular electronic items such as a mobile phone, to be safely carried with safe handling also in possibly hazardous environments.

This object is achieved by a storage device with features as described herein.

Accordingly, the first wall, the second wall, the first strip body and/or the second strip body are wholly or partly fabricated from a composition containing an antimicrobially active additive.

In items of daily life, there can be a need to create safety against microorganisms such as viruses or germs during handling, in order to avoid an infection due to the handling of items. Such a need can exist in particular in an environment possibly loaded by microorganisms, for example in hospitals.

For this reason, it is provided in the proposed storage device to at least sectionally fabricate the storage device from a composition which has antimicrobial properties. In particular, the first wall and/or the second wall of the storage body can be fabricated (completely) from such a composition containing the antimicrobially active additive or be provided with a coating that is fabricated from the composition containing the antimicrobially active additive (and hence partly be fabricated from the composition). Additionally or alternatively, the strip bodies of the closure device can be fabricated completely from the composition containing the antimicrobially active additive or be partly fabricated from such a composition, in that the strip bodies are provided with a coating made of the composition containing the antimicrobially active additive.

Due to the fact that at least portions of the storage body and/or of the closure device have antimicrobial properties, the storage device can have a disinfecting effect on the items received in the storage device. This applies in particular when the storage body is antimicrobially active on the inside of the walls, i.e. on those sides of the walls which face an interior space of the storage body for receiving the item.

Additionally or alternatively, the storage device can also be antimicrobially active on outer surfaces so that when the storage device is being handled, for example when the storage device is grasped by a user, an infection risk is minimized.

By the fact that the additive is antimicrobially active it is meant that the additive can reduce the reproductive capacity or infectivity of microorganisms or can kill or inactivate the same. Antimicrobial activity thus means an activity against microorganisms, for example bacteria or viruses, so that the composition has a disinfecting effect due to the properties of the additive.

The composition for example (in its essential constituent) can consist of a polymer material or a silicone material, to which the antimicrobially active additive is admixed. The composition here can be used to manufacture the first wall, the second wall, the first strip body and/or the second strip body, but can also serve to produce a coating on one or several components of the storage device.

In one embodiment, the additive consists of particles, in particular nanoparticles, containing a silver material, a zinc material, a cobalt material, a cadmium material, a nickel material, a copper material, a titanium dioxide material and/or an organic compound. Particles of a silver material in particular can include or release silver ions and thereby have an antimicrobial effect. What is likewise conceivable, however, are antimicrobial additives, also referred to as additives, based on zinc, based on copper, or on an organic base, such as phenolic biocides, quaternary ammonium compounds (QAC or QUAT) and fungicides (for example thiabendazole).

In one embodiment, the additive can be present as an antimicrobially active glass powder which is made of a mixture containing porous glass particles and an antimicrobial metal material, in particular containing metal ions. Such an additive present as glass powder is described for example in DE 10 2016 003 868 A1.

The production of the glass powder can be effected for example, as described in DE 10 2016 003 868 A1, by the fact that a partial ion exchange is effected at a temperature of 300° C. to 350° C. and an exchange time of 1 to 120 minutes in a mixture which includes
porous glass particles with micropores and macropores from borosilicate glass foamed continuously by means of extrusion with an Fe₂O₃ content<0.2 wt-%, wherein the glass foam obtained subsequently is comminuted by means of dry grinding to mean particle sizes of 1.0 to 8.0 μm,
color stabilizers which contain 0.1% to 0.2% of ammonium ions,
antimicrobial metal ions from dissolved metal salts, wherein the metal ions can be silver ions and/or zinc ions and/or copper ions.

The additive for example can be contained in the composition in an amount of 0.1 wt-% to 20 wt-%. The additive material can be embedded for example in a plastic matrix, in particular a polymer matrix, or a silicone matrix and can be distributed in the composition for example in the form of particles.

In one embodiment, the additive contains at least one photocatalyst generating singlet oxygen when exposed to light. The singlet oxygen generated (in-situ) under the influence of light and the associated oxidative effect provide for an efficient killing of bacteria in particular, but also of viruses, fungi, biofilms and spores. The corresponding additive with the photocatalyst thus at least has an antibacterial effect. For example, the additive Dyphox® can be contained or the additive can be Dyphox®. Dyphox® is sold by TriOptoTec GmbH, Regensburg, Germany, and can be used as a coating or additive.

In one embodiment, the first wall and the second wall each have an inner layer and an outer layer. The inner layer and/or the outer layer here can be fabricated for example from the composition containing the antimicrobially active additive or can have a coating made of the composition containing the antimicrobially active additive. Thus, the first wall and the second wall are formed of (at least) two layers, wherein it is also conceivable and possible that the first wall and the second wall have more than two layers. An inner layer and/or an outer layer here can be fabricated from the described composition and thus can have an antimicrobial effect. When the inner layer is fabricated from the composition or coated with such a composition, an antimicrobial activity is provided in particular in the interior of the storage body. When the outer layer is fabricated from the composition or coated with the composition, an antimicrobial activity is provided in particular on outer surfaces of the storage device.

In one embodiment, the first strip body of the first closure part is enclosed between the inner layer and the outer layer of the first wall. Additionally or alternatively, the second strip body of the second closure part can be enclosed between the inner layer and the outer layer of the second wall. The respective strip body, which can be designed as a massive strip-shaped tape, thus is disposed in an intermediate layer between the inner layer and the outer layer and thus is enclosed between the layers and hence inwardly covered by the inner layer and outwardly covered by the outer layer.

In one embodiment, the first closing portion is formed by the inner layer of the first wall and the second closing portion is formed by the inner layer of the second wall. The closing portion of the respective wall thus is integrally molded to the inner layer of the respective wall. In the closed position, the closing portions flatly rest against each other and produce a sealing termination so that the storage body is sealingly closed towards the outside, in particular so fluid-tightly that no moisture can get into the interior of the storage body.

In one embodiment, the first wall and/or the second wall are flexible. In particular, both the first wall and the second wall can be flexible and pliable so that the storage body can be deformed in a flexible, easily moldable way and thus can adapt in its shape to hold a liquid, a gas or an item. Due to the walls, the storage body thus is designed in the form of a bag in which a liquid, a gas or an item, in particular an electronic item such as a mobile phone or another item of daily life, can be received or be enclosed in a protective way. Thus, such a storage device in particular can serve for the protective transport of a liquid, in particular be designed as a hydration bladder. Such a storage device likewise can be suitable for the transport of a gas.

In one embodiment, the first strip body and/or the second strip body are of elastic design. The first strip body and/or the second strip body here have an increased stiffness with respect to the walls of the storage body, but at the same time are so elastic that they can be bent, in particular about a vertical direction perpendicular to the transverse direction.

The closure parts with their strip bodies serve to provide a (sealing) termination for the storage body via the closing portions so that the storage body is sealingly closed in the closed position. The strip bodies therefor are arranged on the closing portions and thus act on the closing portions, wherein the closure parts cooperate in a magnetically attracting manner and the closing portions thus are magnetically held in contact with each other when the storage device is in its closed position.

To provide magnetic interaction, the first strip body and/or the second strip body can each be formed of a magnetic material, for example in that the strip bodies are formed of a plastic material, in particular a polymer material, or a silicone material to which a magnetic material in the form of magnetic particles is admixed.

Alternatively, the strip bodies can each receive a magnet arrangement of discrete magnetic elements so that the strip bodies cooperate in a magnetically attracting manner by interaction of the magnet arrangements.

It is conceivable that each closure part acts as a permanent magnet, for example in that the strip bodies are formed with permanent magnetic particles and thus are made of a permanent magnetic material, or in that the magnet arrangements of the closure parts are each formed of an arrangement of permanent magnets.

Alternatively, however, it is also possible that one closure part acts as a permanent magnet and the other closure part acts as a ferromagnetic armature, for example in that the ferromagnetically acting closure part has a strip body of a ferromagnetic material (for example of a plastic material to which ferromagnetic particles are admixed) or includes discrete ferromagnetic elements.

When a closure part includes a magnet arrangement of discrete magnetic elements, the discrete magnetic elements for example can be lined up linearly along the transverse direction, wherein the discrete magnetic elements for example can be regularly spaced apart from each other along the transverse direction. It is also conceivable and possible, however, to arrange the discrete magnetic elements in rows and columns with reference to a two-dimensional matrix.

In one embodiment, the closure device—in addition to the first closure part and the second closure part—includes a third closure part that includes a third strip body extended along the transverse direction and is arranged on an offset portion of the first wall, which is different from the first closing portion. In the closed position, the third closure part for example can cooperate with the first closure part in a magnetically attracting manner and for example can serve to hold the first closure part and the second closure part in the closed position in a defined position relative to the storage body.

The offset portion, as seen along the first wall perpendicularly to the transverse direction, in particular is spaced apart from the first closing portion. The third closure part thus is extended parallel to the first closure part on the first wall, but offset transversely to the first closure part.

In the closed position, the third strip body and the first strip body for example can be in flat opposition to each other with surfaces facing each other. The closure parts cooperate in a magnetically attracting manner so that the first closure part is held in a defined positional relation to the third closure part.

In the closed position, due to the hold of the first closure part on the third closure part, the storage body in particular can be folded in a region between the offset portion, on which the third closure part is arranged, and the first closing portion of the first wall, in particular by the walls being bent by 180°, as seen along a path of extension in a cross-section perpendicularly to the transverse direction. This can improve the tightness of the storage body in the closed position.

The object also is achieved by a storage device comprising a storage body for receiving an item, which has a first wall and a second wall, and a magnetic closure device for closing the storage body, which has a first closure part and a second closure part. The first closure part and the second closure part cooperate in a magnetically attracting manner in such a way that in a closed position, in which the storage body is closed, a first closing portion of the first wall and a second closing portion of the second wall rest against each other. It is provided that the first wall, the second wall, the first closure part and/or the second closure part are wholly or partly fabricated from a composition containing an antimicrobially active additive, wherein the additive is an antimicrobially active glass powder that is made of a mixture containing porous glass particles and an antimicrobial metal material.

Such an additive present as glass powder in an initial state before manufacture of the respective component is described for example in DE 10 2016 003 868 A1. The production of the glass powder can be effected for example, as described in DE 10 2016 003 868 A1, by the fact that a partial ion exchange is effected at a temperature of 300 CC to 350° C. and an exchange time of 1 to 120 minutes in a mixture which includes
porous glass particles with micropores and macropores from borosilicate glass foamed continuously by means of extrusion with an Fe₂O₃ content<0.2 wt-%, wherein the glass foam obtained subsequently is comminuted by means of dry grinding to mean particle sizes of 1.0 to 8.0 μm,
color stabilizers which contain 0.1% to 0.2% of ammonium ions,
antimicrobial metal ions from dissolved metal salts, wherein the metal ions can be silver ions and/or zinc ions and/or copper ions.

The additive for example can be contained in the composition in an amount between 0.1 wt-% to 20 wt-%. The additive can be embedded for example in a plastic matrix, in particular a polymer matrix, or a silicone matrix and can be distributed in the composition for example in the form of particles.

With regard to the advantages and advantageous embodiments of this storage device reference is also made to the storage device described above.

### BRIEF DESCRIPTION OF THE DRAWINGS

The idea underlying the solution will be explained in detail below with reference to the exemplary embodiments illustrated in the Figures.

FIG. 1 shows a view of a storage device with a storage body and a closure device according to an exemplary embodiment.

FIG. 2 shows an exploded view of the arrangement of FIG. 1.

FIG. 3A shows a frontal view of the storage device.

FIG. 3B shows a sectional view along line A-A of FIG. 3A.

FIG. 3C shows an enlarged view in section B of FIG. 3B.

FIG. 4 shows a view of a storage device with a storage body and a closure device according to another exemplary embodiment.

FIG. 5 shows an exploded view of the arrangement of FIG. 4.

FIG. 6A shows a frontal view of the storage device.

FIG. 6B shows a sectional view along line C-C of FIG. 6A.

FIG. 6C shows an enlarged view in section C of FIG. 6B.

FIG. 7 shows a view of a storage device with a storage body and a closure device according to yet another exemplary embodiment.

FIG. 8 shows an exploded view of the arrangement of FIG. 7.

FIG. 9A shows a frontal view of the storage device.

FIG. 9B shows a sectional view along line A-A of FIG. 9A.

FIG. 9C shows an enlarged view in section B of FIG. 9B.

### DESCRIPTION OF THE INVENTION

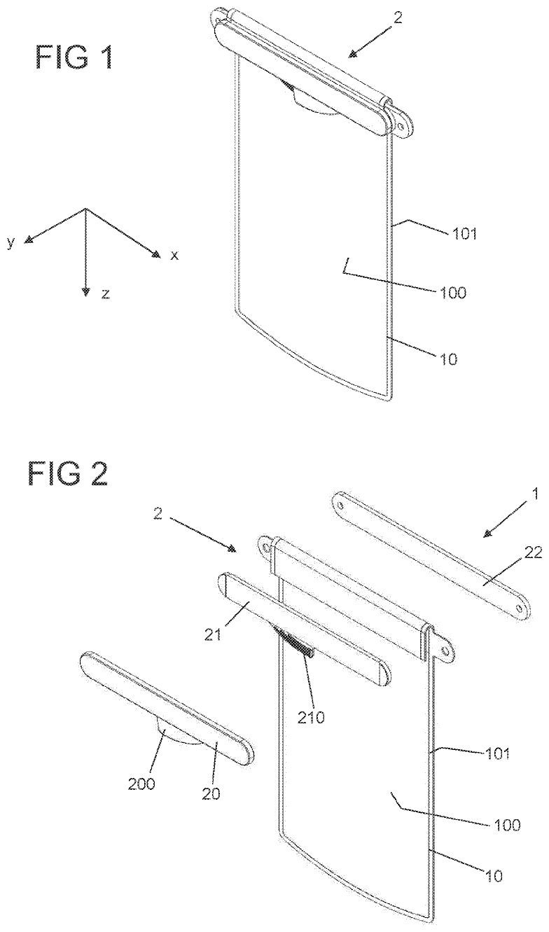

FIGS. 1 to 3C show views of a first exemplary embodiment of a storage device 1 which has a storage body 10 that is formed by walls 100, 101 and is designed to hold a liquid, a gas or an item, in particular an electronic item such as a mobile phone or another item of daily life, for example a purse or a means of payment.

The walls 100, 101 are connected to each other for example by welding on parallel lateral edges spaced apart from each other along a transverse direction X and on a lower edge along a vertical direction Z and are to be closed by a closure device 2 in the region of an upper end such that an inner volume of the storage body 10 is sealingly closed in a closed position of the storage device 1 and an item received therein thus is protected against external influences, in particular moisture and dirt.

The walls 100, 101 are of flexible design so that the storage body 10 is flexibly deformable, in particular in order to be able to flexibly receive an item therein.

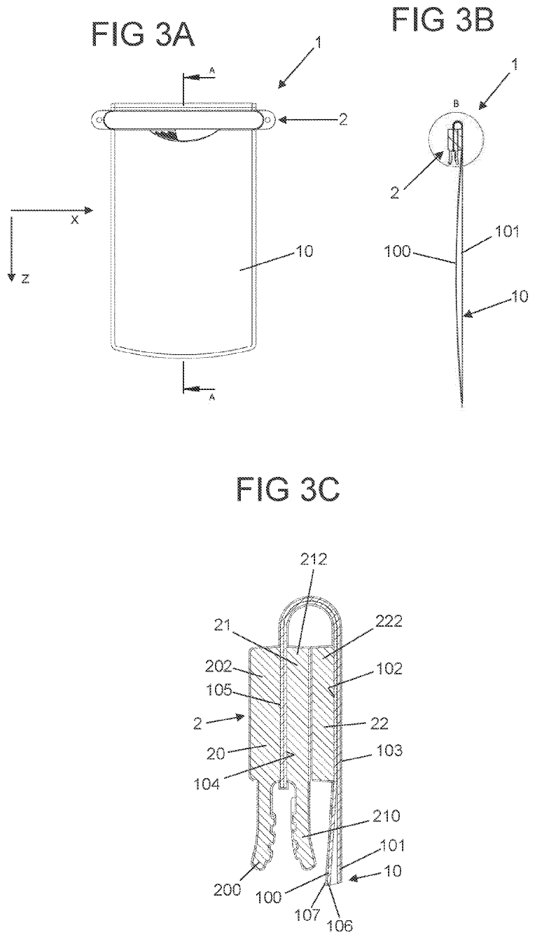

As can be taken from the sectional view of FIG. 3C, each wall 100, 101 is formed by two layers 106, 107, of which an inner layer 106 points towards the inner volume of the storage body 10 and an outer layer 107 points towards the outside.

The closure device 2 includes three closure parts 20, 21, 22.

Of these closure parts 20, 21, 22 a first closure part 21 is arranged on a closing portion 104 of the first wall 100, while a second closure part 20 is arranged on a closing portion 105 of the second wall 101 in such a way that the closure parts 20, 21 extend along the vertical direction Z at the same height on the respectively associated wall 100, 101. The closure parts 20, 21 cooperate in a magnetically attracting manner so that in the closed position of the storage device 1 the closing portions 104, 105, which are each formed by the inner layer 106 of the associated wall 100, 101, flatly and thus sealingly rest against each other.

A third closure part 22 is arranged on a portion 102 of the first wall 100 which is offset from the closing portion 104 of the first wall 100 and, as seen along a path of extension proceeding from the closing portion 104 in a cross-section as shown in FIG. 3C, arranged transversely offset from the first closure part 21.

The closure parts 20, 21, 22 each have a strip body 202, 212, 222 that is elongate along the transverse direction X and thus extends transversely to the vertical direction Z.

The closure parts 20, 21, 22 are designed to cooperate in a magnetically attracting manner. In the illustrated exemplary embodiment, the strip bodies 202, 212, 222 are each formed from a magnetically acting material, for example in that the strip bodies 202, 212, 222 are made of a plastic material or a silicone material into which magnetically active particles are embedded.

The strip bodies 202, 212, 222 can each act as permanent magnets and then face each other in pairs with unlike poles in such a way that in the sequence shown in FIG. 3C the strip bodies 202, 212, 222 magnetically attract each other. Alternatively, for example, merely the strip body 212 of the first closure part 21 can act as a permanent magnet, while the other strip bodies 202, 222 are of the ferromagnetic type so that the associated closure parts 20, 22 act as a magnetic armature. It is also conceivable, for example, that the closure parts 20, 21 are designed to be permanently magnetic at their strip bodies 202, 212, while the strip body 222 of the third closure part 22 acts ferromagnetically.

As can be taken for example from FIG. 3C, the strip bodies 202, 212, 222 are enclosed between the inner layer 106 and the outer layer 107 of the respectively associated wall 100, 101. The strip bodies 212, 222 of the closure parts 21, 22 are enclosed between the inner layer 106 and the outer layer 107 of the first wall 100, while the strip body 202 of the closure part 20 is enclosed between the inner layer 106 and the outer layer 107 of the second wall 101.

The closing portions 104, 105 are urged into flat contact with each other by the first closure part 21 and the second closure part 20 in such a way that the closing portions 104, 105 formed by the inner layers 106 of the walls 100, 101 rest against each other in the manner of membranes and thus sealingly close the storage body 10. The closure parts 20, 21 are held in a defined position by the third closure part 22, wherein a 180° convolution of the walls 100, 101 in a region between the closing portions 104, 105 and portions 102, 103 transversely offset from the closing portions 104, 105 is effected, as this can be taken from FIG. 3C. This further improves the tightness of the closure device 2 in the closed position.

The first closure part 21 and the second closure part 20 each include a handle element 200, 210 which can be grasped by a user. By action on the closure parts 20, 21 the closure parts 20, 21 can be released in particular from the further, third closure part 22 and be moved away from each other along an opening direction Y, which is directed perpendicularly to the transverse direction X and to the vertical direction Z, so that access to the interior of the storage body 10 is possible.

In the illustrated exemplary embodiment at least parts of the storage device 1 are fabricated from an antimicrobially active material or are provided with an antimicrobially active coating. The material consists of a composition which contains an antimicrobially active additive, for example silver-based, zinc-based, copper-based, cobalt-based, cadmium-based particles, particles based on titanium dioxide or particles based on an organic compound. In one embodiment, the antimicrobially active material is a material which contains a photocatalyst generating singlet oxygen (in-situ) when exposed to light. For example, the antimicrobially active material is a material which contains Dyphox®. The material can be used for manufacturing parts of the storage device as such, but can also serve to produce a coating on one or several parts.

For example, the walls 100, 101 can wholly or partly be fabricated from such a material. For example, the inner layer 106 and/or the outer layer 107 of each wall 100, 101 can be fabricated from such a material. For example, when the inner layer 106 of one or both walls 100, 101 is fabricated from a material containing an antimicrobially active additive, the storage device 1 has an antimicrobial effect on items received in the storage body 10. When the outer layer 107 additionally or alternatively is fabricated from a material containing an antimicrobially active additive, the storage device 1 has an antimicrobial effect on its outer surfaces.

The material of the inner layer 106 and of the outer layer 107 here can be the same or different.

Additionally or alternatively, the strip bodies 202, 212, 222 can also be fabricated from an antimicrobially active material.

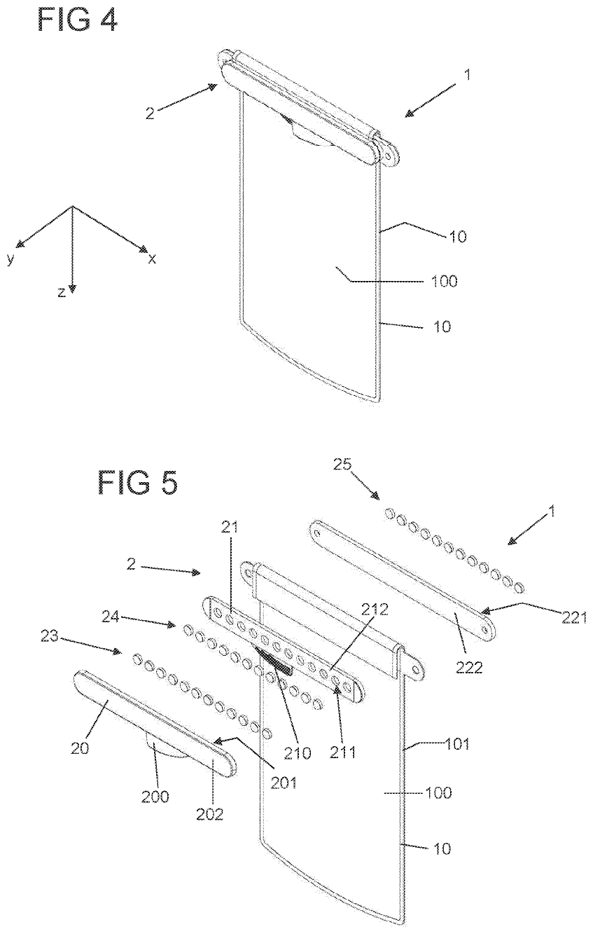

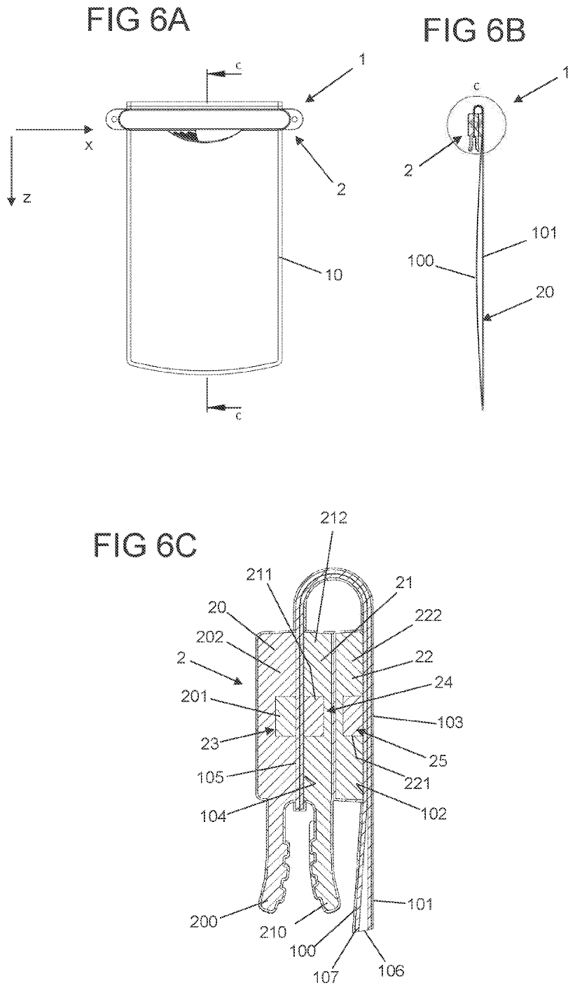

In an exemplary embodiment shown in FIGS. 4 to 6C, the strip bodies 202, 212, 222 are not entirely fabricated from a magnetic material, but each include an arrangement of receiving openings 201, 211, 221 in which discrete magnetic elements of one magnet arrangement 23, 24, 25 each are received. The discrete magnetic elements are lined up one beside the other along the transverse direction X and thus form a linear array of magnetic elements, wherein in the closed position the magnet arrangements 23, 24, 25 of the different closure parts 20, 21, 22 cooperate in a magnetically attracting manner and thus sealingly close the storage body 10 via the closing portions 104, 105.

The discrete magnetic elements of the magnet arrangements 23, 24, 25 can each be formed by discrete permanent magnets, for example from a neodymium material. It is also conceivable, however, that merely one magnet arrangement 23, 24, 25 or two of the magnet arrangements 23, 24, 25 include discrete permanent magnets, while the other magnet arrangements 23, 24, 25 are formed of discrete ferromagnetically active elements.

Otherwise, the exemplary embodiment of FIGS. 4 to 6C is functionally equivalent to the exemplary embodiment of FIGS. 1 to 3C, in particular also with regard to the manufacture of parts of the storage device 1 from a material containing an antimicrobially active additive so that in this respect reference fully is to be made to the preceding explanations.

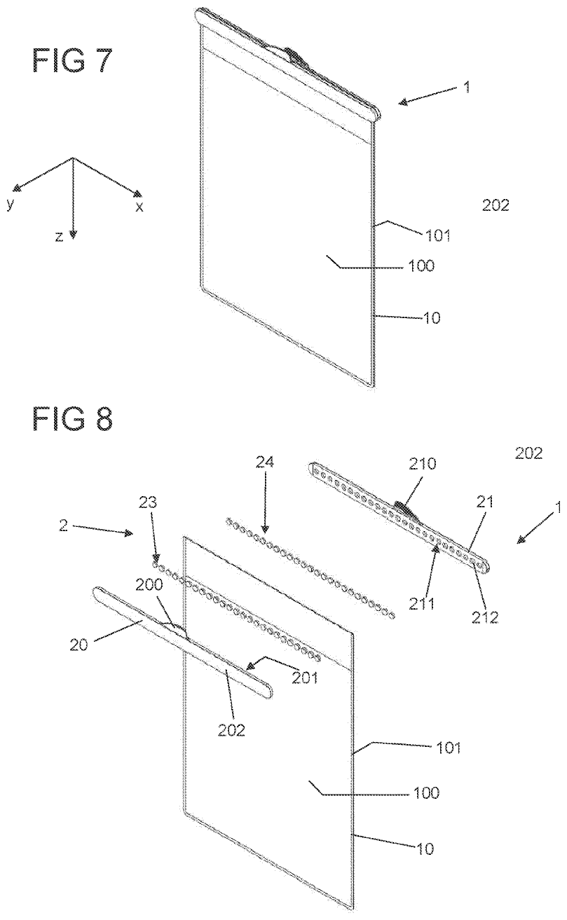

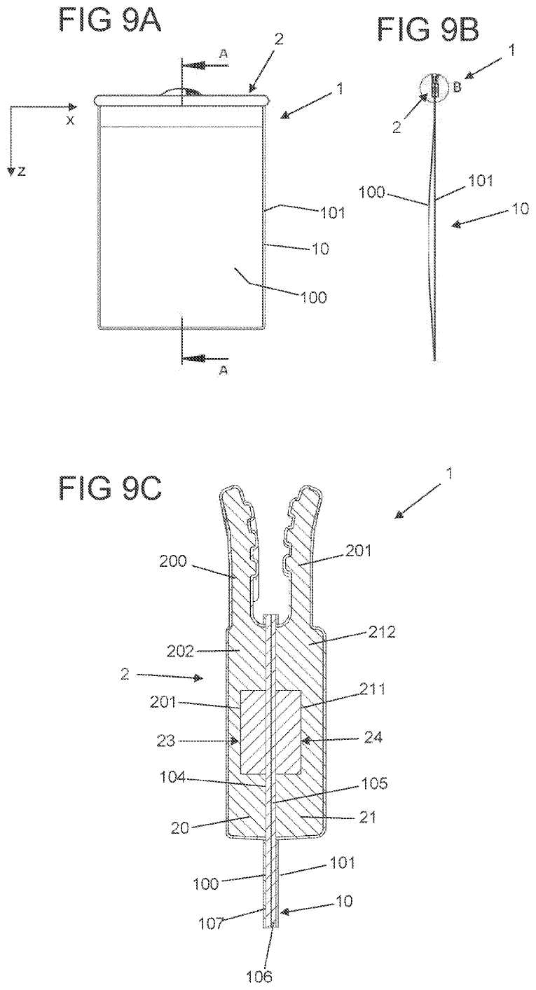

The exemplary embodiment of FIGS. 7 to 9C corresponds to the exemplary embodiment of FIGS. 4 to 6C, wherein in this case there is no third closure part 22, like in the exemplary embodiment of FIGS. 4 to 6C, and the closure device 2 thus is formed by merely two closure parts 20, 21. Otherwise, the storage device 1 is functionally equivalent to the storage device 1 of FIGS. 4 to 6C.

The idea underlying the solution is not limited to the exemplary embodiments described above, but can also be realized in an entirely different way.

In particular, for example materials with an antimicrobially active additive, which in particular have an antibacterial and antiviral effect, other than the materials mentioned here are also conceivable and possible.

### LIST OF REFERENCE NUMERALS

1 storage device
10 storage body
100, 101 wall
102, 103 portion
104, 105 closing portion
106 inner layer
107 outer layer
2 closure device
20, 21, 22 closure part
200, 210 handle element
201, 211, 221 receiving openings
202, 212, 222 strip body
23, 24, 25 magnet arrangement
X, Y, Z direction

1. A storage device, comprising a storage body for holding a liquid, a gas or an item, which has a first wall and a second wall, and a magnetic closure device for closing the storage body, which has a first closure part and a second closure part, wherein the first closure part has a first strip body elongate along a transverse direction and is arranged on a first closing portion of the first wall and wherein the second closure part has a second strip body elongate along the transverse direction and is arranged on a second closing portion of the second wall, wherein the first closure part the second closure part cooperate in a magnetically attracting manner in such a way that in a closed position, in which the storage body is closed, the first closing portion and the second closing portion rest against each other, and
wherein at least one of the first wall, the second wall, the first strip body and the second strip body are wholly or partly fabricated from a composition containing an antimicrobially active additive.

2. The storage device according to claim 1, wherein at least one of at least one layer of the first wall, at least one layer of the second wall, the first strip body and the second strip body is fabricated from the composition containing the antimicrobially active additive or has a coating which is fabricated from the composition containing the antimicrobially active additive.

3. The storage device according to claim 1, wherein the composition contains a polymer material or a silicone material to which the antimicrobially active additive is admixed.

4. The storage device according to any of claim 1, wherein the additive includes particles, in particular at least one of nanoparticles, containing a silver material, a zinc material, a cobalt material, a cadmium material, a nickel material, a copper material and a titanium dioxide material.

5. The storage device according to claim 1, wherein the additive is an antimicrobially active glass powder which is produced from a mixture containing porous glass particles and an antimicrobial metal material.

6. The storage device according to claim 1, wherein the additive contains at least one photocatalyst generating singlet oxygen when exposed to light.

7. The storage device according to claim 1, wherein the additive is contained in the composition in an amount of 0.1 wt-% to 20 wt-%.

8. The storage device according to claim 1, wherein the first wall and the second wall each have an inner layer and an outer layer, and wherein at least one of the inner layer and the outer layer are fabricated from the composition containing the antimicrobially active additive or have a coating of the composition containing the antimicrobially active additive.

9. The storage device according to claim 8, wherein at least one of the first strip body is enclosed between the inner layer and the outer layer of the first wall and the second strip body is enclosed between the inner layer and the outer layer of the second wall.

10. The storage device according to claim 8, wherein the first closing portion is formed by the inner layer of the first wall and the second closing portion is formed by the inner layer of the second wall.

11. The storage device according to claim 1, wherein in the closed position the first closing portion and the second closing portion sealingly rest against each other.

12. The storage device according to claim 1, wherein at least one of the first wall and the second wall are flexible.

13. The storage device according to claim 1, wherein at least one of the first strip body and the second strip body are elastic.

14. The storage device according to claim 1, wherein at least one of the first strip body and the second strip body are molded from a magnetic material or include a magnet arrangement formed of discrete magnetic elements.

15. The storage device according to claim 1, and wherein the closure device includes a third closure part which has a third strip body extended along the transverse direction and is arranged on an offset portion of the first wall which is different from the first closing portion, wherein in the closed position the third closure part cooperates with the first closure part in a magnetically attracting manner.

16. The storage device according to claim 15, wherein in the closed position the third strip body and the first strip body are opposite each other with their surfaces facing each other.

17. The storage device according to claim 15, wherein in the closed position the storage body is folded in a region between the offset portion and the first closing portion.

18. A storage device, comprising a storage body for holding a liquid or an item, which has a first wall and a second wall, and a magnetic closure device for closing the storage body, which has a first closure part and a second closure part, wherein the first closure part and the second closure part cooperate in a magnetically attracting manner in such a way that in a closed position, in which the storage body is closed, a first closing portion of the first wall and a second closing portion of the second wall rest against each other,
wherein at least one of the first wall, the second wall, the first closure part and the second closure part are wholly or partly fabricated from a composition containing an antimicrobially active additive, wherein the additive is an antimicrobially active glass powder that is made from a mixture containing porous glass particles and an antimicrobial metal material.
